(19) United States
(12) Patent Application Publication
(10) Pub. No.: US 2023/0414506 A1
(43) Pub. Date: Dec. 28, 2023

(54) PEPTIDE FORMULATIONS AND METHODS OF USE

(71) Applicant: REALTA LIFE SCIENCES, INC., Norfolk, VA (US)

(72) Inventors: Neel K. KRISHNA, Norfolk, VA (US); Kenji CUNNION, Norfolk, VA (US)

(73) Assignee: REALTA LIFE SCIENCES, INC., Norfolk, VA (US)

(21) Appl. No.: 18/035,846

(22) PCT Filed: Nov. 3, 2021
(86) PCT No.: PCT/US2021/057802

§ 371 Date: May 8, 2023

Related U.S. Application Data
(60) Provisional application No. 63/111,367, filed on Nov. 9, 2020.

(51) Int. Cl. A61K 9/107; A61K 47/12; A61K 47/20; A61K 38/10; A61K 9/00

(52) U.S. Cl. CPC A61K 9/1075; A61K 47/12; A61K 47/20; A61K 38/10; A61K 9/0019

(57) ABSTRACT

The present invention provides synthetic peptides. The invention is directed to modifications of a synthetic peptide of 15 amino acids from the Polar Assortant (PA) peptide, which is a scrambled peptide derived from human Astrovirus protein. In some embodiments, the invention is directed to pharmaceutical formulations of the peptides including lipid-based formulations and formulations suitable for intravenous administration.

### CROSS-REFERENCE TO RELATED APPLICATIONS

This application claims priority to U.S. Provisional Application No. 63/111,367, filed on 9 Nov. 2020, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 25, 2021, is named 251110_000157_SL.txt and is 1,169 bytes in size.

### BACKGROUND OF THE INVENTION

### 1. Field of the Invention

Embodiments of the present invention relates generally to synthetic peptides and uses thereof for therapy and diagnostics, and more specifically to pharmaceutical formulations of the peptides including lipid-based formulations and formulations suitable for intravenous administration.

### 2. Background

The Complement System

The complement system, an essential component of the innate immune system, plays a critical role as a defense mechanism against invading pathogens, primes adaptive immune responses, and helps remove immune complexes and apoptotic cells. Three different pathways comprise the complement system: the classical pathway, the lectin pathway and alternative pathway. C1q and mannose-binding lectin (MBL) are the structurally related recognition molecules of the classical and lectin pathways, respectively. Whereas IgM or clustered IgG serve as the principal ligands for C1q, MBL recognizes polysaccharides such as mannan. Ligand binding by C1q and MBL results in the sequential activation of C4 and C2 to form the classical and lectin pathway C3-convertase, respectively. In contrast, alternative pathway activation does not require a recognition molecule, but can amplify C3 activation initiated by the classical or lectin pathways. Activation of any of these three pathways results in the formation of inflammatory mediators (C3a and C5a) and the membrane attack complex (MAC), which causes cellular lysis.

While the complement system plays a critical role in many protective immune functions, complement activation is a significant mediator of tissue damage in a wide range of autoimmune and inflammatory disease processes. (Ricklin and Lambris, “Complement-targeted therapeutics.” Nat Biotechnol 2007; 25(11):1265-75).

Naturally occurring peptides are essential signaling molecules that play critical physiological roles in human biology in the form of neurotransmitters, hormones, growth factors and anti-microbials [1]. Given their intrinsic specificity and efficient properties, this class of molecules have received considerable attention as human therapeutics for a variety of disease indications, with over 60 approved for therapeutic use in the US, Europe and/or Japan and 155 currently in clinical development as of March, 2018 [2]. The advantageous properties of peptides provides a significant advantage over small molecules (<500 Da) which often suffer from toxicity and off-target effects. Additionally, compared to large protein-based molecules such as humanized monoclonal antibodies, peptides typically enjoy low costs of manufacturing and in many cases can be synthesized chemically, thus avoiding costly and complex production and purification. Often, naturally occurring peptides cannot be directly translated into therapeutic use due to sub-optimal chemical and physical stability and poor pharmacokinetics (half-life). Thus, a number of technological approaches to rationally design peptides into more druggable molecules suitable for human administration are frequently employed.

A need exists for complement regulators. On the one hand, the complement system is a vital host defense against pathogenic organisms. On the other hand, its unchecked activation can cause devastating host cell damage. Currently, despite the known morbidity and mortality associated with complement dysregulation in many disease processes, including autoimmune diseases such as systemic lupus erythematosus, myasthenia gravis, and multiple sclerosis, only two anti-complement therapies have recently been approved for use in humans: 1) eculizumab (Soliris™) and 2) ultomiris (Ravulizumab™) two humanized, long-acting monoclonal antibodies against C5 used in the treatment of paroxysmal nocturnal hemoglobinuria (PNH) and atypical hemolytic uremic syndrome (aHUS). PNH and aHUS are orphan diseases in which very few people are afflicted. Currently, no complement regulators are approved for the more common disease processes in which dysregulated complement activation plays a pivotal role. Dysregulated complement activation can play a role in both chronic disease indications and acute disease indications.

Developing peptides to inhibit classical, lectin and alternative pathways of the complement system is needed, as each of these three pathways have been demonstrated to contribute to numerous autoimmune and inflammatory disease processes. Specific blockade of classical and lectin pathways is particularly needed, as both of these pathways have been implicated in ischemia reperfusion-induced injury among other diseases in many animal models. Humans with alternative pathway deficiencies suffer severe bacterial infections. Thus, a functional alternative pathway is essential for immune surveillance against invading pathogens.

The inventors have identified a novel family of peptides known as PIC1 (also referred to as EPICC peptides). The PIC1 peptides possess multiple anti-inflammatory properties including inhibition of the classical pathway of complement, myeloperoxidase (MPO) inhibition, neutrophil extracellular trap (NET) inhibition as well as intrinsic antioxidant and anti-microbial activity [3-8]. The precursor to the PIC1 peptides were initially based upon the finding that the 787 amino acid capsid protein sequence of human astrovirus type 1, a non-enveloped icosahedral RNA virus that is an endemic pathogen causing gastroenteritis in human infants [9], could inhibit activation of the classical pathway of complement [10].

The PIC1/EPICC family of molecules comprise a collection of rationally designed peptides with several anti-inflammatory functional properties including inhibition of the classical pathway of complement, myeloperoxidase inhibition, neutrophil extracellular trap inhibition and antioxidant activity. The original PIC1 peptide is a 15 amino acid peptide sequence, IALILEPICCQERAA (SEQ ID NO: 1), derived from a scrambled astroviral coat protein. The original PIC1 peptide has been modified with a C-terminal monodisperse 24-mer PEGylated moiety (IALILEPICCQERAA-dPEG24; PA-dPEG24; SEQ ID NO: 2), increasing its aqueous solubility. A sarcosine substitution scan of SEQ ID NO: 2 revealed that replacement of isoleucine at position 8 with sarcosine resulted in a peptide, IALILEP(Sar)CCQERAA (PA-I8Sar; SEQ ID NO: 3) that was water soluble without PEGylation (as described in U.S. Pat. No. 10,005,818). Solubility studies showed that the PA-I8Sar peptide was amphipathic. Different formulations of the PA-I8Sar peptide were developed, including lipid-based formulations and formulations suitable for intravenous administration. Different formulations of PA-dPEG24 were also developed, including formulations suitable for intravenous administration.

### BRIEF SUMMARY OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to pharmaceutically useful formulations of the synthetic peptides, including for example and not limitation, lipid-based formulations of the synthetic peptides, particularly peptides that are present in or associated with lipid micelles, and formulations that are suitable for intravenous administration.

In one aspect, the present invention provides a composition comprising a therapeutically effective amount of SEQ ID NO: 3 and a lipid-based carrier. In some embodiments, the lipid-based carrier comprises lipid micelles. In some embodiments, the lipid-based carrier comprises a lipid emulsion, e.g., Intralipid® carrier. In some embodiments, the Intralipid® carrier is present in an amount of about 10% w/v to about 20% w/v.

In a related aspect, the present invention provides a composition comprising a therapeutically effective amount of SEQ ID NO: 2 or SEQ ID NO: 3 and at least one excipient. In some embodiments, the at least one excipient is suitable for intravenous administration. In some embodiments, the at least one excipient is selected from the group consisting of a citrate, an ascorbate, amino acids, and combinations thereof. In some embodiments, the citrate comprises sodium citrate. In some embodiments, the citrate is present in an amount of about 1% w/v to about 5% w/v. In some embodiments, the ascorbate comprises sodium ascorbate. In some embodiments, the ascorbate is present in an amount of about 1% w/v to about 5% w/v. In some embodiments, the amino acids comprise L-methionine. In some embodiments, the amino acids are present in an amount of about 0.01% w/v to about 5% w/v.

In some embodiments of any of the compositions described herein, SEQ ID NO: 2 or SEQ ID NO: 3 is present in an amount of about 0.001 to about 200 milligrams per kilogram (mg/kg) of body weight. In some embodiments of any of the compositions described herein, SEQ ID NO: 2 and/or SEQ ID NO: 3 is present in an amount of about 5 to about 160 mg/kg. In some embodiments of any of the compositions described herein, SEQ ID NO: 2 or SEQ ID NO: 3 is present in an amount of about 1 mg/ml to about 100 mg/ml. In some embodiments of any of the compositions described herein, SEQ ID NO: 2 or SEQ ID NO: 3 is present in an amount of about 10 mg/ml to about 80 mg/ml.

In a related aspect, the present invention provides a method of altering cytokine expression comprising administering to a subject in need thereof any of the compositions described herein. In some embodiments, the administration comprises parenteral administration. In some embodiments, the administration comprises intravenous administration.

In a related aspect, the present invention provides a method of treating or preventing a disease or condition comprising administering to a subject in need thereof any of the compositions described herein. In some embodiments, the administration comprises parenteral administration. In some embodiments, the administration comprises intravenous administration.

These and other objects, features and advantages of the present invention will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1 shows that PA-I8Sar (also referred to herein as RLS-0088) formulated in Intralipid® (also referred to herein as Lipo-RLS-0088) inhibited complement activation to the same degree as RLS-0088 formulated in histidine buffer. Values are represented as a percent of the positive control, which consists of human O sera and AB red blood cells in GVBS⁺⁺ buffer. Data shown are mean of n=4±Standard error of the mean.

FIGS. 2A-2B show that intravenous (IV) administration of Lipo-RLS-0088 inhibited complement activation with a higher functionality over time relative to RLS-0088. Male Wistar rats with indwelling jugular catheters were administered 200 mg/kg RLS-0088 in Intralipid® or RLS-0088 in histidine buffer at 200 mg/kg of the compound as a single, bolus, IV infusion. At various time points after infusion (0.5, 2, 5, 60, 120, and 240 minutes), an aliquot of blood was drawn and plasma isolated and frozen at −70 C pending analysis. The C1q binding assay was then performed. FIG. 2A shows the results of the C1q binding assay with a Y-axis maximum value of 6 mg/ml RLS-0088, while FIG. 2B shows the same results with a Y-axis maximum value of 0.8 mg/ml RLS-0088.

FIGS. 3A-3B shows the effect of different excipients on the inhibition of hemolytic activity of RLS-0071 (3A) and RLS-0088 (3B) in the ABO incompatibility CH50-type assay. Data are the means of n=3 independent experiments+ SEM.

FIG. 4 shows the appearance of different RLS-0088 formulations, where F denotes a freeze-thawed formulation and L denotes a lyophilized formulation.

FIG. 5 shows the appearance of different RLS-0071 formulations, where F denotes a freeze-thawed formulation and L denotes a lyophilized formulation.

### DETAILED DESCRIPTION OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to pharmaceutically useful formulations of the synthetic peptides, including for example and not limitation, lipid-based formulations of the synthetic peptides, particularly peptides that are present in lipid micelles, and formulations that are suitable for intravenous administration.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

As used herein, the term “combination” of a synthetic peptide according to the claimed invention and at least a second pharmaceutically active ingredient means at least two, but any desired combination of compounds can be delivered simultaneously or sequentially (e.g., within a 24 hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “analog” or “functional analog” refers to a related modified form of a polypeptide, wherein at least one amino acid substitution, deletion, or addition has been made such that said analog retains substantially the same biological activity as the unmodified form, in vivo and/or in vitro.

The terms “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

Several different computer programs are available to determine the degree of identity between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at www.accelrys.com/products/gcg), using either a Blosum 62 matrix or a PAM250 matrix, and a gap weight of 16, 14, 12, 10, 8, 6, or 4 and a length weight of 1, 2, 3, 4, 5, or 6. These different parameters will yield slightly different results but the overall percentage identity of two sequences is not significantly altered when using different algorithms.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

“Sequence identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95%, e.g., at least 96%, 97%, 98%, 99%, or 100% “sequence identity” to a reference nucleotide sequence, it is intended that the nucleotide sequence of the given polynucleotide is identical to the reference sequence except that the given polynucleotide sequence may include up to 5, 4, 3, 2, 1, or 0 point mutations per each 100 nucleotides of the reference nucleotide sequence. In other words, in a polynucleotide having a nucleotide sequence having at least 95%, e.g., at least 96%, 97%, 98%, 99%, or 100% sequence identity relative to the reference nucleotide sequence, up to 5%, 4%, 3%, 2%, 1%, or 0% of the nucleotides in the reference sequence may be deleted or substituted with another nucleotide, or a number of nucleotides up to 5%, 4%, 3%, 2%, 1%, or 0% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95%, e.g., at least 96%, 97%, 98%, 99%, or 100% sequence identity to a reference amino acid sequence, it is intended that the given amino acid sequence of the polypeptide is identical to the reference sequence except that the given polypeptide sequence may include up to 5, 4, 3, 2, 1, or 0 amino acid alterations per each 100 amino acids of the reference amino acid sequence. In other words, to obtain a given polypeptide sequence having at least 95%, e.g., at least 96%, 97%, 98%, 99%, or 100% sequence identity with a reference amino acid sequence, up to 5%, 4%, 3%, 2%, 1%, or 0% of the amino acid residues in the reference sequence may be deleted or substituted with another amino acid, or a number of amino acids up to 5%, 4%, 3%, 2%, 1%, or 0% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

As used herein, the term “immune response” includes innate immune responses, T-cell mediated immune responses, and/or B-cell mediated immune responses. Exemplary immune responses include T cell responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes (e.g. neutrophils).

“Parenteral” administration of an immunogenic composition includes, e.g., subcutaneous (s.c.), intravenous (i.v.), intramuscular (i.m.), or intradermal (i.d.) injection, or infusion techniques.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

A “variant” of a polypeptide according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Within the meaning of the present invention, the term “conjoint administration” is used to refer to administration of a composition according to the invention and another therapeutic agent simultaneously in one composition, or simultaneously in different compositions, or sequentially (preferably, within a 24 hour period).

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Peptide Compositions of the Invention

Modifications of the amino acid structure of CP1 has led to the discovery of additional peptides that are able to regulate complement activation, such as C1q activity. It was previously demonstrated that modifications such as PEGylation enhanced solubility of the peptides as well as potent inhibition of biological activity compared to the parent molecule (IALILEPICCQERAA; SEQ ID NO: 1) in in vitro assays of classical complement pathway activation/inhibition, myeloperoxidase (MPO) inhibition, antioxidant activity and inhibition of NET activity. A peptide with a C-terminal monodisperse 24-mer PEGylated moiety was found to be highly soluble and had strong inhibition of the complement system (IALILEPICCQERAA-dPEG24; SEQ ID NO: 2; PA-dPEG24). A sarcosine substitution of the non-PEGylated peptide was found to have similar solubility to the PEGylated peptide (IALILEP(Sar)CCQERAA; SEQ ID NO: 3; PA-I8Sar). Different formulations of PA-I8Sar were explored in order to develop formulations suitable for intravenous administration, including lipid-based formulations and formulations with excipients including citrate (e.g., trisodium citrate dihydrate), ascorbate (e.g., sodium ascorbate), and/or amino acids (e.g., L-methionine). Formulations of PA-dPEG24 with excipients including citrate (e.g., trisodium citrate dihydrate), ascorbate (e.g., sodium ascorbate), and/or amino acids (e.g., L-methionine) were also developed.

The term “peptide(s),” as used herein, refers to amino acid sequences, which may be naturally occurring, or peptide mimetics, peptide analogs and/or synthetic derivatives (including for example but not limitation PEGylated peptides) of about 15 amino acids based on SEQ ID NO: 2. In addition, the peptide may be less than about 15 amino acid residues, such as between about 10 and about 15 amino acid residues and such as peptides between about 5 to about 10 amino acid residues. Peptide residues of, for example, 5, 6, 7, 8, 9, 10, 11, 12, 13, 14, and 15 amino acids are equally likely to be peptides within the context of the present invention. Peptides can also be more than 15 amino acids, such as, for example, 16, 17, 18, 19, and 20, or more amino acids.

The disclosed peptides can be formulated in lipid-based carriers or with excipients suitable for intravenous administration. The lipid-based carrier can be Intralipid®. Intralipid® can be used for parenteral nutrition supplementation in humans to increase caloric intake intravenously and is composed of an emulsion of lipid micelles. The peptides can be associated with the lipid micelles, e.g., integrated into a layer of the lipid micelle. The excipients suitable for intravenous administration can comprise citrate (e.g., sodium citrate or sodium citrate dihydrate), ascorbate (e.g., sodium ascorbate), or amino acids (e.g., L-methionine).

Substitutes for an amino acid within the peptide sequence may be selected from other members of the class to which the amino acid belongs. For example, the nonpolar (hydrophobic) amino acids include alanine, leucine, isoleucine, valine, proline, phenylalanine, tryptophan, and methionine. Amino acids containing aromatic ring structures include phenylalanine, tryptophan, and tyrosine. The polar neutral amino acids include glycine, serine, threonine, cysteine, tyrosine, asparagine, and glutamine. The positively charged (basic) amino acids include arginine and lysine. The negatively charged (acidic) amino acids include aspartic acid and glutamic acid. For example, one or more amino acid residues within the sequence can be substituted by another amino acid of a similar polarity, which acts as a functional equivalent, resulting in a silent alteration.

A conservative change generally leads to less change in the structure and function of the resulting protein. A non-conservative change is more likely to alter the structure, activity, or function of the resulting protein. For example, the peptide of the present disclosure comprises one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Particularly preferred amino acid substitutions include:
a) Ala for Glu or vice versa, such that a negative charge may be reduced;
b) Lys for Arg or vice versa, such that a positive charge may be maintained;
c) Ala for Arg or vice versa, such that a positive charge may be reduced;
d) Glu for Asp or vice versa, such that a negative charge may be maintained;
e) Ser for Thr or vice versa, such that a free —OH can be maintained;
f) Gln for Asn or vice versa, such that a free NH2 can be maintained;
g) Ile for Leu or for Val or vice versa, as roughly equivalent hydrophobic amino acids;
h) Phe for Tyr or vice versa, as roughly equivalent aromatic amino acids; and
i) Ala for Cys or vice versa, such that disulfide bonding is affected.

Substitutes for an amino acid within the peptide sequence may be selected from any amino acids, including, but not limited to alanine, arginine, asparagine, aspartic acid, cysteine, glutamic acid, glutamine, glycine, histidine, isoleucine, leucine, lysine, methionine, phenylalanine, proline, pyrolysine, selenocysteine, serine, threonine, tryptophan, tyrosine, valine, N-formyl-L-methionine, sarcosine, or other N-methylated amino acids. In some embodiments, sarcosine substitutes for an amino acid within the peptide sequence.

In one aspect, the present invention provides lipid-based formulations of synthetic peptides that regulate the complement system and methods of using these peptides. Specifically, in some embodiments, the lipid-based formulations of the synthetic peptides can bind, regulate and inactivate C1 and MBL, and therefore can efficiently inhibit classical and lectin pathway activation at its earliest point while leaving the alternative pathway intact. These lipid-based formulations of peptides are of therapeutic value for selectively regulating and inhibiting C1 and MBL activation without affecting the alternative pathway and can be used for treating diseases mediated by dysregulated activation of the classical and lectin pathways. In other embodiments, the lipid-based formulations of peptides regulate classical pathway activation but not lectin pathway activation. The lipid-based formulations of peptides are useful for various therapeutic indications, including for example and not limitation, diseases of organs with high lipid content such as the brain and pancreas (e.g., hypoxic ischemic encephalopathy (HIE), stroke, traumatic brain injury, pancreatitis). The inventive lipid-based formulations may also be used for subcutaneous depot dosing for chronic inflammatory conditions, such as for example and not limitation, lupus, antineutrophil cytoplasmic antibody-associated vasculitis (ANCA vasculitis), Behcet's disease, autoimmune nephritis, and nephropathies.

In one aspect, the present invention provides intravenous formulations of synthetic peptides that regulate the complement system and methods of using these peptides. Specifically, in some embodiments, the intravenous formulations of the synthetic peptides can bind, regulate and inactivate C1 and MBL, and therefore can efficiently inhibit classical and lectin pathway activation at its earliest point while leaving the alternative pathway intact. These intravenous formulations of peptides are of therapeutic value for selectively regulating and inhibiting C1 and MBL activation without affecting the alternative pathway and can be used for treating diseases mediated by dysregulated activation of the classical and lectin pathways. In other embodiments, the intravenous formulations of peptides regulate classical pathway activation but not lectin pathway activation. The intravenous formulations of peptides are useful for various therapeutic indications.

In one embodiment, the invention provides synthetic peptides derived from human astrovirus coat protein, the peptides comprising the amino acid sequences and modifications of SEQ ID NO: 2, such as for example and not limitation, sarcosine substitutions of SEQ ID NO: 2. In one embodiment, the invention provides lipid-based formulations of any of the peptides discussed herein, including for example but not limitation, SEQ ID NO: 3. In some embodiments, the lipid-based formulation comprises lipid micelles. In some embodiments, the lipid-based formulation comprises Intralipid®.

In one embodiment, the invention provides formulations of any of the peptides discussed herein, including for example but not limitation, SEQ ID NO: 3, that are suitable for intravenous administration. In one embodiment, the invention provides intravenous pharmaceutical formulations of any of the peptides discussed herein, including for example but not limitation, SEQ IS NO: 2 and/or SEQ ID NO: 3, further comprising additional excipients suitable for intravenous administration, such as for example and not limitation, citrate (e.g., trisodium citrate dihydrate), ascorbate (e.g., sodium ascorbate), and/or amino acids (e.g., L-methionine).

TABLE 1
List of Peptides of the Invention.

| SEQ | | |
|---|---|---|
| ID | | |
| NO. | Sequence | Description |
| 1 | IALILEPICCQERAA | PA (PIC1) |
| 2 | IALILEPICCQERAA-PEG24 | PA-dPEG24 |
| 3 | IALILEP(Sar)CCQERAA | PA-I8Sar |

In other embodiments, the synthetic peptides are capable of altering cytokine expression. In some embodiments, the invention provides a method of altering cytokine expression comprising administering to the subject in need thereof a composition comprising a therapeutically effective amount of a lipid-based formulation of a synthetic peptide comprising SEQ ID NO: 3. In some embodiments, the lipid-based formulation comprises lipid micelles. In some embodiments, SEQ ID NO: 3 is present within the lipid micelles. In some embodiments, the lipid-based formulation comprises Intralipid® and SEQ ID NO: 3.

In some embodiments, the invention provides a method of altering cytokine expression comprising intravenously administering to the subject in need thereof a pharmaceutical formulation comprising a therapeutically effective amount of a synthetic peptide comprising SEQ ID NO: 2 or SEQ ID NO: 3. In some embodiments, the pharmaceutical formulation can comprise excipients, carriers, and other ingredients that are suitable for intravenous administration. In some embodiments, the pharmaceutical formulation comprises a therapeutically effective amount of SEQ ID NO: 2 or SEQ ID NO: 3 and a citrate (such as for example and not limitation, sodium citrate or sodium citrate dihydrate). In one embodiment, the excipient is an antioxidant that is suitable for use in adult subjects (e.g., citrate, trisodium citrate, trisodium citrate trihydrate). In one embodiment, the excipient is an antioxidant that is suitable for use in pediatric or infant subjects (e.g., ascorbate such as sodium ascorbate, and/or amino acids such as L-methionine). In some embodiments, the citrate can be present in an amount of about 1% w/v to about 5% w/v, including about 1% w/v to about 2.5% w/v. In some embodiments, the pharmaceutical formulation comprises a therapeutically effective amount of SEQ ID NO: 2 or SEQ ID NO: 3 and an ascorbate (such as for example and not limitation, sodium ascorbate). In some embodiments, the ascorbate can be present in an amount of about 1% w/v to about 5% w/v, including about 2.5% w/v to about 4.5% w/v. In some embodiments, the pharmaceutical formulation comprises a therapeutically effective amount of SEQ ID NO: 2 or SEQ ID NO: 3 and an amino acid (such as for example and not limitation, L-methionine). In some embodiments, the amino acid can be present in an amount of 0.01% w/v to about 5% w/v, including about 0.1% w/v to about 1% w/v.

The disclosed peptides can selectively regulate C1q and MBL activation without affecting alternative pathway activity and are, thus, ideal for preventing and treating diseases mediated by the dysregulated activation of the classical and lectin pathways. Specific blockade of classical and lectin pathways are particularly needed, as both of these pathways have been implicated in ischemia-reperfusion induced injury in many animal models. [Castellano et al., “Therapeutic targeting of classical and lectin pathways of complement protects from ischemia-reperfusion-induced renal damage.” Am J Pathol. 2010; 176(4):1648-59; Lee et al., “Early complement factors in the local tissue immunocomplex generated during intestinal ischemia/reperfusion injury.” Mol. Immunol. 2010 February; 47(5):972-81; Tjernberg, et al., “Acute antibody-mediated complement activation mediates lysis of pancreatic islets cells and may cause tissue loss in clinical islet transplantation.” Transplantation. 2008 Apr. 27; 85(8):1193-9; Zhang et al. “The role of natural IgM in myocardial ischemia-reperfusion injury.” J Mol Cell Cardiol. 2006 July; 41(1):62-7). The alternative pathway is essential for immune surveillance against invading pathogens, and humans with alternative pathway defects suffer severe bacterial infections. By binding and inactivating C1q and MBL, the peptides can efficiently regulate classical and lectin pathway activation while leaving the alternative pathway intact.

The term “regulate,” as used herein, refers to i) controlling, reducing, inhibiting or regulating the biological function of an enzyme, protein, peptide, factor, byproduct, or derivative thereof, either individually or in complexes; ii) reducing the quantity of a biological protein, peptide, or derivative thereof, either in vivo or in vitro; or iii) interrupting a biological chain of events, cascade, or pathway known to comprise a related series of biological or chemical reactions. The term “regulate” may thus be used, for example, to describe reducing the quantity of a single component of the complement cascade compared to a control sample, reducing the rate or total amount of formation of a component or complex of components, or reducing the overall activity of a complex process or series of biological reactions, leading to such outcomes as cell lysis, formation of convertase enzymes, formation of complement-derived membrane attack complexes, inflammation, or inflammatory disease. In an in vitro assay, the term “regulate” may refer to the measurable change or reduction of some biological or chemical event, but the person of ordinary skill in the art will appreciate that the measurable change or reduction need not be total to be “regulatory.”

In some embodiments, the present invention relates to therapeutically active peptides having the effects of regulating the complement system.

### Pharmaceutical Compositions of the Invention

The present disclosure provides pharmaceutical compositions capable of regulating the complement system, comprising at least one peptide, as discussed above, and at least one pharmaceutically acceptable carrier, diluent, stabilizer, or excipient. Pharmaceutically acceptable carriers, excipients, or stabilizers are nontoxic to recipients at the dosages and concentrations employed. They can be solid, semi-solid, or liquid. The pharmaceutical compositions of the present invention can be in the form of tablets, pills, powders, lozenges, sachets, cachets, elixirs, suspensions, emulsions, solutions, or syrups.

The pharmaceutical compositions of the present invention are prepared by mixing the peptide having the appropriate degree of purity with pharmaceutically acceptable carriers, diluents, or excipients. Examples of formulations and methods for preparing such formulations are well known in the art. The pharmaceutical compositions of the present invention are useful as a prophylactic and therapeutic agent for various disorders and diseases, as set forth above. In one embodiment, the composition comprises a therapeutically effective amount of the peptide. In another embodiment, the composition comprises at least one other active ingredient effective in regulating the complement system. In another embodiment, the composition comprises at least one other active ingredient effective in treating at least one disease associated with the complement system. In another embodiment, the composition comprises at least one other active ingredient effective in treating at least one disease that is not associated with the complement system. The term “therapeutically effective amount,” as used herein, refers to the total amount of each active component that is sufficient to show a benefit to the subject.

The therapeutically effective amount of the peptide varies depending on several factors, such as the condition being treated, the severity of the condition, the time of administration, the route of administration, the rate of excretion of the peptide employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

The effective daily dose generally is within the range of from about 0.001 to about 200 milligrams per kilogram (mg/kg) of body weight, including about 5 to about 160 mg/kg, about 10 to about 160 mg/kg, about 40 mg/kg to about 160 mg/kg, and about 40 mg/kg to about 100 mg/kg. This dose can be achieved through a 1-6 time(s) daily dosing regimen. The effective dosage generally is within the range of about 1 mg/ml to about 100 mg/ml, including about 10 mg/ml to about 80 mg/ml. The effective dosage may depend on the additional ingredients included in the pharmaceutical composition. Alternatively, optimal treatment can be achieved through a sustained release formulation with a less frequent dosing regimen. The effective dosage may depend on the age of the subject. For example and not limitation, a pediatric or infant subject may require a lower dosage of the pharmaceutical composition than an adult subject. An effective dosage for a pediatric or infant subject may be about 10 mg/ml. An effective dosage for an adult subject may be about 80 mg/ml.

In another aspect, the invention provides a pharmaceutical composition comprising a therapeutically effective amount of a lipid-based formulation of SEQ ID NO: 3 and at least one pharmaceutically acceptable carrier, diluent, or excipient.

In another aspect, the invention provides an intravenous pharmaceutical formulation comprising a therapeutically effective amount of SEQ ID NO: 2 or SEQ ID NO: 3 and at least one pharmaceutically acceptable carrier, diluent, or excipient that is suitable for intravenous administration. In one embodiment, the excipient can comprise citrate (e.g., trisodium citrate dihydrate), ascorbate (e.g., sodium ascorbate), and/or amino acids (e.g., L-methionine). In one embodiment, the excipient is an antioxidant that is suitable for use in adult subjects (e.g., citrate, trisodium citrate, trisodium citrate trihydrate). In one embodiment, the excipient is an antioxidant that is suitable for use in pediatric or infant subjects (e.g., ascorbate such as sodium ascorbate, and/or amino acids such as L-methionine).

The compositions of the invention can comprise a carrier and/or excipient. While it is possible to use a compound of the present invention for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Oral formulations readily accommodate additional mixtures. Solid dosage forms for oral administration can also be used and can include, e.g., capsules, tablets, caplets, pills, troches, lozenges, powders, and granules. Non-limiting examples of suitable excipients include, e.g., diluents, buffering agents (e.g., sodium bicarbonate), preservatives, stabilizers, binders, compaction agents, lubricants, dispersion enhancers, disintegration agents, antioxidants, flavoring agents, sweeteners, and coloring agents. Those of relevant skill in the art are well able to prepare suitable solutions.

In one embodiment of any of the compositions of the invention, the composition is formulated for delivery by a route such as, e.g., oral, topical, rectal, mucosal, sublingual, nasal, naso/oro-gastric gavage, parenteral, intraperitoneal, intradermal, transdermal, intrathecal, nasal, and intracheal administration. In one embodiment of any of the compositions of the invention, the composition is in a form of a liquid, foam, cream, spray, powder, or gel. In one embodiment of any of the compositions of the invention, the composition comprises a buffering agent (e.g., sodium bicarbonate).

Administration of the compounds and compositions in the methods of the invention can be accomplished by any method known in the art. Non-limiting examples of useful routes of delivery include oral, rectal, fecal (by enema), and via naso/oro-gastric gavage, as well as parenteral, intraperitoneal, intradermal, transdermal, intrathecal, nasal, and intracheal administration. The active agent may be systemic after administration or may be localized by the use of regional administration, intramural administration, or use of an implant that acts to retain the active dose at the site of implantation.

The useful dosages of the compounds and formulations of the invention can vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses may be effective to achieve a therapeutic effect. While it is possible to use a compound of the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice. Although there are no physical limitations to delivery of the formulations of the present invention, oral delivery is preferred for delivery to the digestive tract because of its ease and convenience, and because oral formulations readily accommodate additional mixtures, such as milk, yogurt, and infant formula.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

Solutions or suspensions can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity, such as sodium chloride or dextrose.

In instances in which the agents exhibit insufficient solubility, methods for solubilizing agents may be used. Such methods are known to those of skill in this art, and include, but are not limited to, using co-solvents, such as, e.g., dimethylsulfoxide (DMSO), using surfactants, such as TWEEN® 80, or dissolution in aqueous sodium bicarbonate. Pharmaceutically acceptable derivatives of the agents may also be used in formulating effective pharmaceutical compositions.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Parenteral administration, generally characterized by injection, either subcutaneously, intramuscularly or intravenously, is also contemplated herein. Injectables can be prepared in conventional forms, either as liquid solutions or suspensions, solid forms suitable for solution or suspension in liquid prior to injection, or as emulsions. Suitable excipients include, by way of example and without limitation, water, saline, dextrose, glycerol or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate and cyclodextrins.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution can be apportioned into vials for lyophilization. Each vial can contain, by way of example and without limitation, a single dosage (10-1000 mg, such as 100-500 mg) or multiple dosages of the agent. The lyophilized powder can be stored under appropriate conditions, such as at about 4° C. to room temperature. Reconstitution of this lyophilized powder with water for injection provides a formulation for use in parenteral administration.

Combination Therapies

A further embodiment of the invention provides a method of regulating the complement system, comprising administering to a subject a pharmaceutical formulation of the present invention. While the pharmaceutical formulations of the present invention can be administered as the sole active pharmaceutical agent, they can also be used in combination with one or more therapeutic or prophylactic agent(s) that is(are) effective for regulating the complement system. In this aspect, the method of the present invention comprises administrating a pharmaceutical formulation of the present invention before, concurrently, and/or after one or more additional therapeutic or prophylactic agents effective in regulating the complement system.

The pharmaceutical formulations of the present invention can be administered with additional agent(s) in combination therapy, either jointly or separately, or by combining the pharmaceutical formulations and the additional agent(s) into one composition. The dosage is administered and adjusted to achieve maximal regulation of the complement system. For example, both the pharmaceutical formulations and the additional agent(s) are usually present at dosage levels of between about 10% and about 150%, more preferably, between about 10% and about 80%, of the dosage normally administered in a mono-therapy regimen.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Lipid-based Formulation of PA-I8Sar

PA (IALILEPICCQERAA; SEQ ID NO: 1) was solubilized in 20% Intralipid®, which resulted in a homogeneous solution. Upon centrifugation, PA separated into lipid and aqueous layers. Interestingly, at a concentration of 2 mg/ml, 60% of PA was associated with the lipid layer, while only 40% of PA remained in the aqueous phase. If this mixture was allowed to sit at 4° C. for 48 hours, the mixture became paste-like and it had enough viscosity that it could be applied topically.

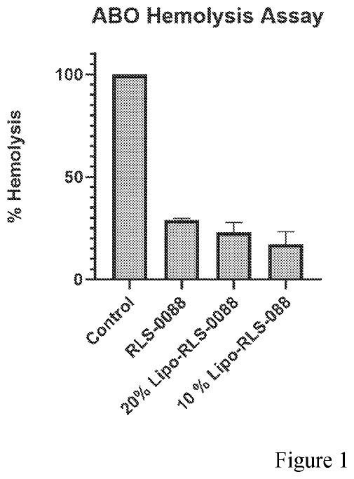

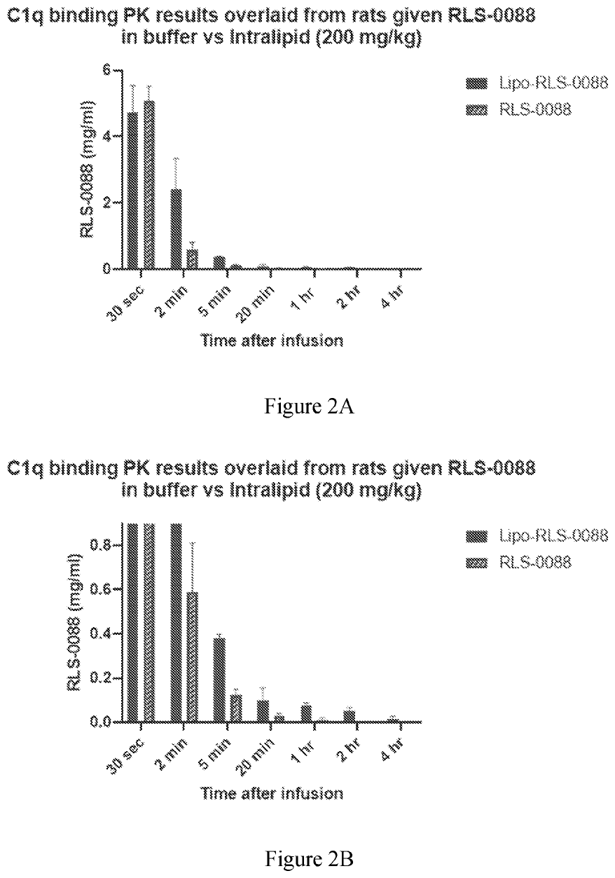

When PA-dPEG24 (RLS-0071; SEQ ID NO: 2) or PA-I8Sar (RLS-0088; SEQ ID NO: 3) were solubilized in 20% Intralipid®, or if they were mixed in their soluble form 1:1 with 20% Intralipid® (resulting in 10% Intralipid®), they both resulted in a homogeneous solution which does not separate well upon centrifugation. The ability of lipo-RLS-0088 to inhibit complement activation was tested in in vitro assays and it was surprisingly found that the ability to inhibit complement activation was retained and similar to RLS-0088 in solution (FIG. 1). This was unexpected given that in lipo-RLS-0088 the active pharmacologic agent is associated with the micelles, yet was able to interact with and inhibit activation of hydrophilic complement components in solution. Given that the in vitro activity was preserved, the inhibiting effects of lipo-RLS-0088 was tested in vivo by IV injection into adult rats. The control was identical doses of RLS-0088 in solution injected into a different set of adult rats. The pharmacodynamics showed a 4-fold increase in functionality over time (measured by C1q binding assay) for the rats receiving IV lipo-RLS-0088 compared with those receiving the same dose of IV RLS-0088 (FIGS. 2A-2B).

### Methods

### ABO Hemolysis Assay

For hemolytic complement assays, human red blood cells (RBCs) from type AB donors were purified, washed, and standardized to 1.0×10 9 cells/ml. Human sera from type O donors at a 10% final concentration was combined with 1.0 mM of the peptides and the volume was brought up to 0.250 ml with GVBS' and 5.0×10 7 RBCs. The samples were incubated for 1 hour at 37° C. and then spun at 3,000 rpm for 5 minutes and the supernatant was collected and read at 412 nm.

### In Vivo Testing

Male Wistar rats with indwelling jugular catheters were administered 200 mg/kg RLS-0088 in Intralipid® or RLS-0088 in histidine buffer at 200 mg/kg of the compound as a single, bolus, IV infusion. At various time points after infusion (0.5, 2, 5, 60, 120, and 240 minutes), an aliquot of blood was drawn and plasma isolated and frozen at −70° C. pending analysis. Animals were sacrificed after the terminal blood draw and a gross necropsy performed. The C1q binding assay was then performed.

### C1q Binding Assay

An Immunlon-2 HB ELISA plate was coated with 1 μg/ml C1q in bicarbonate buffer overnight at 4° C. The plates were washed with PBS-T (phosphate buffered saline+0.1% Tween) and then blocked with 1% gelatin/PBS for 2 hours at room temperature. After washing, the plates were incubated with the plasma samples diluted in 1% gelatin/PBS for 1 hour at room temperature followed by washing. Plates were then probed with rabbit antibody raised against the peptide IALILEPICCQERAA (SEQ ID NO: 1) that lacked PEGylation (PA) at 1:1000 in 1% gelatin/PBS for 1 hour at room temperature followed by a goat anti-rabbit HRP (Sigma Aldrich, St Louis, MO) at 1:1,000 in 1% gelatin/PBS for 1 hour at room temperature with a washing step in between. After addition of TMB substrate solution to the wells, the reaction was stopped using 1N H2SO4, and the plate read on a BioTek Synergy HT plate reader at 450 nm.

### Example 2: Intravenous Formulations of PA-I8Sar

PA-dPEG24 (RLS-0071) and PA-I8Sar (RLS-0088) have been successfully formulated in liquid containing histidine buffer. To develop formulations of these two molecules that can be utilized for intravenous administration to pediatric and adult patients, the following excipients were utilized to formulate PA-dPEG24 and PA-I8Sar:
Citrate (trisodium citrate dihydrate) at 2.5% w/v for adult formulation
Ascorbate (sodium ascorbate) at 2.88% w/v for infant formulation
L-Methionine buffer at 0.16% w/v for infant formulation

For the citrate buffer conditions, PA-dPEG24 and PA-I8Sar were formulated at a concentration of 80 mg/ml at pH 6.8. For PA-dPEG24 it was noted that the preparation would start to gel if not kept on ice. This gelling behavior was not observed for PA-I8Sar.

For the ascorbate and L-methionine conditions, PA-dPEG24 and PA-I8Sar were formulated at a concentration of 10 mg/ml at pH 6.8.

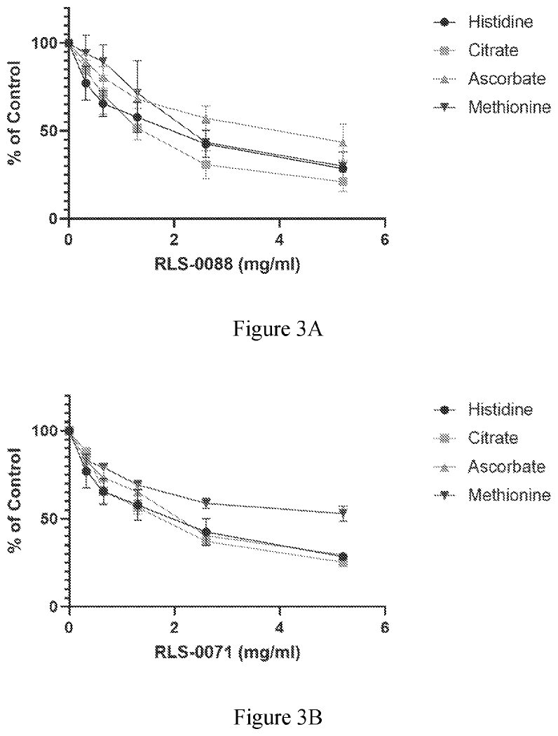

To ascertain if these various excipients had any effect on the functional activity of PA-dPEG24 and PA-I8Sar, the formulated peptides were tested in a hemolytic assay to determine the ability of these molecules to inhibit complement activity. For hemolytic complement assays, human red blood cells (RBCs) from type AB donors were purified, washed and standardized to 1×10⁹ cells/ml, as previously described. Human sera from type O donors at a 20% final concentration was combined with increasing concentrations of the formulated peptides and the volume was brought up to 0.15 ml with GVBS++ buffer and 0.5 ml RBCs. The samples were spun at 3,000 rpm for 5 minutes and the supernatant was collected and read at 412 nm. Values are represented as a percent of the positive control, which consists of human O sera and AB red blood cells in GVBS++ buffer without peptide addition. PA-dPEG24 and PA-I8Sar formulated in citrate, ascorbate and methionine excipients were able to maintain dose-dependent inhibition of complement activation in the hemolytic assay compared to the original histidine formulated molecules (FIGS. 3A-3B). Under these experimental conditions, PA-dPEG24 formulated in ascorbate, citrate or histidine had slightly increased complement inhibiting activity compared to peptide formulated in methionine (FIG. 3A). In the case of PA-I8Sar, formulations comprising each of the four excipients demonstrated similar levels of activity (FIG. 3B). These findings demonstrated that PA-dPEG24 and PA-I8Sar may be efficiently solubilized in four different excipients that maintain the functional activity of these peptides.

### Example 4: Preparation of a Stable Liquid Formulation for RLS-0088

### Materials

TABLE 2
Composition Table

| | Formulation ID (%, wt) |
|---|---|
| Ingredient | F32 |
| RLS-0088 | 4.0 |
| Methionine | 0.48 |
| Trehalose | 5.0 |
| Hydroxypropyl-β-cyclodextrin (HP-β-CD) | 10.0 |
| EDTA•Na2 | 0.05 |
| DI water* | 80.47 |
| pH | 3.0 |

*Amount needs to be corrected by the purity and water content of RLS-0088.

TABLE 3
Compounding Table

| | Formulation ID (g) |
|---|---|
| Ingredient | F32 |
| RLS-0088* | 0.216 |
| Methionine | 0.024 |
| Trehalose | 0.250 |
| Hydroxypropyl-β-cyclodextrin (HP-β-CD) | 0.500 |
| EDTA•Na2 | 0.0025 |
| DI water* | 4.008 |
| Adjust pH with 10N NaOH or 1N HCl | 3.0 |
| Batch Size (g) | 5.0 |

*RLS-0088 amount = pure RLS-0088/(purity %)/(100% − water content %) = 0.2 g/(95.5%)/(100% − 2.9%) = 0.216 g.

### Protocol

### Preparation of Vehicles for Formulations (F32V)

The F32V vehicles were prepared in 50 mL Falcon tubes according to Table 4 as follows. The tubes were vortexed to dissolve all solids.

TABLE 4
F32V vehicles

| Ingredient | F32V |
|---|---|
| Methionine | 0.096 |
| Trehalose | 1.000 |
| Hydroxypropyl-β-cyclodextrin (HP-β-CD) | 2.000 |
| EDTA•Na2 | 0.010 |
| DI water* | 16.031 |

### Preparation of Formulations (F32)

RLS-0088 was weighed out and transferred into 15 mL Falcon tubes for F32. The QS to batch size using corresponding vehicle was prepared according to Table 5 below.

TABLE 5

| Sample ID | F32 |
|---|---|
| RLS-0088 (g) | 0.216 |
| F32V (g) | 4.784 |
| pH after Adjustment | 3.02 |
| Batch size (g) | 5.000 |

Each tube was vortexed to dissolve the solids. NaOH or HCl solution was used to adjust each sample to the target pH values. The solution was filtered by 0.2 μm nylon filter. The filtrate was transferred into 2 HPLC vials, one was kept in 25° C. chamber and another was kept in 2-8° C. chamber. The filtrate was assayed at T0 and 24 h. At 24 h, the particle size of filtrate was checked by Accusizer. For the remaining formulation, 0.8 mL/vial of formulation was transferred into four 2 mL lipolyzing glass vials. The vials were frozen −20° C. After freezing, two of the vials were lyophilized. The frozen and lyophilized vials were melted and reconstituted and kept at 2-8° C. or 25° C. The samples were tested according to Table 6.

TABLE 6
Testing protocol

| | | Treatment | | | |
|---|---|---|---|---|---|
| | | Frozen F32 at −20° C. | | Lyophilized F32 | |
| | | Storage Temp. after melting or recon. | | | |
| | | 2-8° C. | 25° C. | 2-8° C. | 25° C. |
| Testing | T 0 | Assay | Assay | Assay | Assay |
| | 24 h | — | Assay, | — | Assay, |
| | | | particulate | | particulate |
| | | | matter*, pH | | matter*, pH |
| | 48 h | Assay, | — | Assay, | — |
| | | particulate | | particulate | |
| | | matter*, pH | | matter*, pH | |

*Tested by Accusizer.

A HPLC sample was prepared by transferring 100 mg of each sample into a 10 mL volumetric flask. QS to 5 mL by the diluent.

### Results.

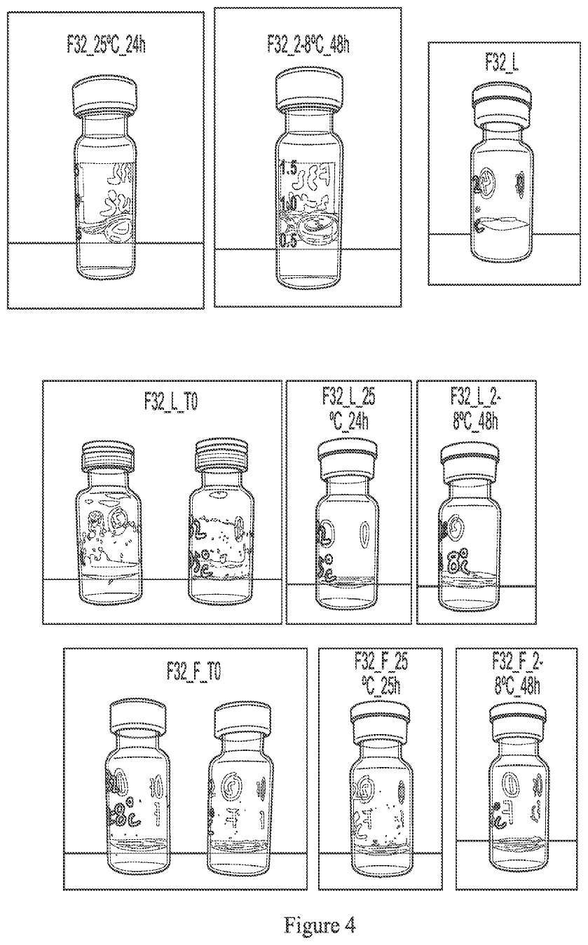

FIG. 4 shows the vials of formulations containing RLS-0088 under the different storage conditions. Table 7 below shows the Accusizer results.

TABLE 7
Accusizer results

| | Particle | Particle | Particle |
|---|---|---|---|
| Sample ID | Size (μm) | number/mL | number/35 mL* |
| F32_25° C._24 h | 10 | 137 | 4785 |
| | 25 | 17 | 598 |
| F32_2-8° C._48 h | 10 | 171 | 5981 |
| | 25 | 34 | 1196 |
| F32_L_25° C._24 h | 10 | 68 | 2392 |
| | 25 | 17 | 598 |
| F32_L_2-8° C._48 h | 10 | 68 | 2392 |
| | 25 | 0 | 0 |
| F32_F_25° C._24 h | 10 | 85 | 2990 |
| | 25 | 17 | 598 |
| F32_F_2-8° C._48 h | 10 | 68 | 2392 |
| | 25 | 0 | 0 |

| | |
|---|---|
| Acceptance criteria (USP 26 <788>) | 10 μm Counts <= 6000 per 1 Container |
| | 25 μm Counts <= 600 per 1 Container |

*Based on 1400 mg/70 kg human; 1400 mg/(40 mg/mL) = 35 mL.
Note:
F: Freeze-thaw;
L: lyophilized

All F32 samples, regardless of whether freeze-thawed or lyophilized-reconstituted, met or were close to the particulate matter acceptance criteria in USP 26 <788> after storing at 25° C. for 24 and 2-8° C. for 48 h.

HPLC Results

Sample preparation was performed according to Table 8.

TABLE 8
HPLC samples

| | API | | Vehicle | Target | | |
|---|---|---|---|---|---|---|
| | weight | | weight | Conc. | | |
| Sample | (mg) | Vehicle | (g) | (mg/g)* | pH_T0 | Appearance |
| F32 | 216.5 | F32V | 4.776 | 40.2 | 3.1 | Slightly cloudy with |
| | | | | | | no visible particles |
| | | | | | | at 25° C. after 24 h |
| | | | | | | and clear/hazy at |
| | | | | | | 2-8° C. after 48 h. |

Target conc. is the theoretical assay when all solids are dissolved = API weight * purity * (100% − water content)/(Vehicle weight + API weight)

Stability results are presented in Table 9 below.

TABLE 9
Stability results

| | | | | | API conc. | QS to | | | |
|---|---|---|---|---|---|---|---|---|---|
| | Sample | PA of | PA of | Ave. | in Diluent | Volume | Assay | Recovery | Purity |
| Sample ID (pH) | Size (mg) | 1st Inj. | 2nd Inj. | PA | (mg/mL) | (mL) | (mg/g) | % vs. T0 | % |
| F32_T0 | 102.5 | 12931 | 13120 | 13026 | 0.854 | 5 | 41.7 | — | 95.98 |
| F32_25° C._24 h | 95.8 | 12278 | 12259 | 12268 | 0.804 | 5 | 42.0 | 100.7 | 95.75 |
| F32_2-8° C._48 h | 102.5 | 13130 | 13162 | 13146 | 0.850 | 5 | 41.5 | 99.6 | 96.55 |
| F32_L_25° C._T0 | 96.7 | 11830 | 11831 | 11831 | 0.766 | 5 | 39.6 | — | 93.32 |
| F32_L_25° C._24 h | 100.0 | 12109 | 12182 | 12145 | 0.787 | 5 | 39.4 | 99.3 | 92.33 |
| F32_L_2-8° C._T0 | 99.5 | 12049 | 12019 | 12034 | 0.780 | 5 | 39.2 | — | 93.43 |
| F32_L_2-8° C._48 h | 101.4 | 12315 | 12287 | 12301 | 0.797 | 5 | 39.3 | 100.4 | 93.24 |
| F32_F_25° C._T0 | 99.0 | 12551 | 12548 | 12550 | 0.813 | 5 | 41.1 | — | 95.59 |
| F32_F_25° C._24 h | 100.0 | 12718 | 12715 | 12716 | 0.824 | 5 | 41.2 | 100.3 | 95.19 |
| F32_F_2-8° C._T0 | 97.6 | 12371 | 12346 | 12359 | 0.801 | 5 | 41.0 | — | 95.83 |
| F32_F_2-8° C._48 h | 101.7 | 13170 | 12981 | 13076 | 0.847 | 5 | 41.7 | 101.5 | 95.90 |

Note:
F: Freeze-thaw;
L: lyophilize

The F32 formulations were stable at 25° C. for at least 24 h and 2-8° C. for at least 48 h. The lyophilized-reconstituted and freeze-thaw Formulation F32 were all stable at 25° C. for 24 h and 2-8° C. for 48 h.

### Osmolality

TABLE 10
Osmolality.

| Sample | Osmolality (mOsm) |
|---|---|
| F32 (fresh prepared) | 411 (no dilution) |
| F32 (25° C._24 h) | 169 (after being diluted 1:1 v/v by DI water) * |
| F32V | 316 (no dilution) |

The Osmometer failed to measure osmolality of F32 formulation without dilution. Thus, the sample was diluted 1:1 v/v by DI water for measurement. The osmolality of F32 is 411 mOsm, which is acceptable for IV injection/infusion.

### CONCLUSIONS

The F32 formulation was stable at 25° C. for at least 24 h and 2-8° C. for at least 48 h. The lyophilized-reconstituted and freeze-thaw Formulation F32 were all stable at 25° C. for 24 h and 2-8° C. for 48 h. All F32 samples, regardless of whether they were freeze-thaw or lyophilized-reconstituted, met the particulate matter acceptance criteria in USP 26 <788> after storing at 25° C. for 24 and 2-8° C. for 48 h. The osmolality of F32 is 411 mOsm, which is acceptable for IV injection/infusi on.

### Example 5: Preparation of a Stable Liquid and Lyophilized Pediatric Formulation for RLS-0071

The objective of this example was to prepare a stable liquid and lyophilized formulation of RLS-0071 for pediatric use by: (i) preparing F7, F8, F9, F10 formulations, (ii) lyophilizing the formulations in vials using an existing lyophilize cycle, and (iii) reconstituting the lyophile and thaw the frozen vials and test assay, impurity and particulate matter.

### Materials

Tables 11 and 12 show the composition of the different formulations.

TABLE 11
Composition Table.

Formulation ID (%, wt)

| Ingredient | F7 | F8 | F9 | F10 |
|---|---|---|---|---|
| RLS-0071 | 1.0 | 1.0 | 1.0 | 1.0 |
| Methionine | — | 0.25 | 0.25 | 0.25 |
| EDTA•Na₂ | — | 0.05 | 0.05 | 0.05 |
| Trehalose | 10.0 | 8.7 | 8.2 | — |
| Sucrose | — | — | — | 7.6 |
| Buffer 1 (Citric acid) | — | — | 90.5 | 91.1 |
| WFI | 89.0 | 90.0 | — | — |

| | |
|---|---|
| pH adjusts | 5.3 |

TABLE 12
Compounding Table

| | Formulation ID (mg/40 g) | | | |
|---|---|---|---|---|
| Ingredient | F7 | F8 | F9 | F10 |
| RLS-0071 | 400.0 | 400.0 | 400.0 | 400.0 |
| Methionine | — | 100.0 | 100.0 | 100.0 |
| EDTA•Na₂ | — | 20.0 | 20.0 | 20.0 |
| Trehalose | 4000.0 | 3480.0 | 3280.0 | — |
| Sucrose | — | — | — | 3040.0 |
| Buffer 1 (Citric acid) | — | — | QS to 40 g | QS to 40 g |
| WFI | QS to 40 g | QS to 40 g | — | — |

| | |
|---|---|
| pH adjusts | 5.3 |

### Protocol

Formulations (F7-F10) were prepared as follows. 400 mg of RLS-0071 was weighed out and transferred into falcon tubes for F7-F10. Methionine, EDTA. Na2, Trehalose and sucrose were weighed out and transferred into corresponding falcon tubes. Formulations were brought to 40 g using WFI or Buffer. Each vial was vortexed to dissolve the solids. NaOH or HCl solution was used to adjust each sample to the target pH values.

The formulations were then lyophilized as follows: F7-F10 were filtered by 0.2 μm filter. 4 mL of each filtrate was aliquoted to 10 mL clean lipolyzing glass vials. The glass vials were lyophilized using an existing lyophilization cycle. The lyophilized vials were then reconstituted, and the reconstituted and thawed solutions were stored at 2-8° C. for 12-24 h and 25° C. for 2 h. The solutions were then assayed for particulate matter and impurities.

### Results.

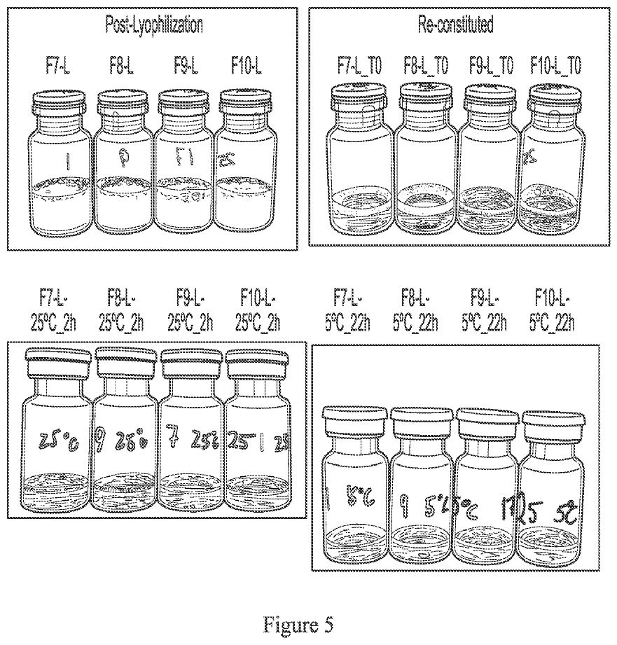

FIG. 5 shows the vials of formulations containing RLS-0071 under the different storage conditions. Tables 13 and 14 below shows the HPLC and particulate matter results.

TABLE 13
HPLC results.

| | | Cal. | Recovery | Degradation | | | Osmolality |
|---|---|---|---|---|---|---|---|
| Sample ID | | Con.(mg/mL) | (%) | (%) | Appearance | pH | (mOsm) |
| F7 | T0 | 9.80 | 100.00 | 0.00 | Clear | 5.31 | 331 |
| | Lyo_T0 | 9.77 | 99.65 | 0.35 | Clear | — | — |
| | Lyo-25° C._2 h | 9.70 | 99.00 | 1.00 | Clear | 5.29 | — |
| | Lyo-5° C._22 h | 9.71 | 99.04 | 0.96 | Clear | — | — |
| F8 | T0 | 9.78 | 100.00 | 0.00 | Clear | 5.29 | 315 |
| | Lyo_T0 | 9.66 | 98.80 | 1.20 | Clear | — | — |
| | Lyo-25° C._2 h | 9.71 | 99.33 | 0.67 | Clear | 5.30 | — |
| | Lyo-5° C._22 h | 9.77 | 99.90 | 0.10 | Clear | — | — |
| F9* | T0 | 7.59 | 100.00 | 0.00 | Precipitate (Un-fil) | 5.30 | 314 |
| | | | | | Clear (Post-fil) | | |
| | Lyo_T0 | 7.45 | 98.15 | 1.85 | Clear | — | — |
| | Lyo-25° C._2 h | 7.41 | 97.63 | 2.37 | Less particles | 5.29 | — |
| | Lyo-5° C._22 h | 7.38 | 97.18 | 2.82 | Clear | — | — |
| F10 | T0 | 9.88 | 100.00 | 0.00 | Clear | 5.30 | 323 |
| | Lyo_T0 | 9.71 | 98.28 | 1.72 | Clear | — | — |
| | Lyo-25° C._2 h | 9.77 | 98.85 | 1.15 | Clear | 5.19 | — |
| | Lyo-5° C._22 h | 9.81 | 99.23 | 0.77 | Clear | — | — |

TABLE 14
Particulate matter

| ID | F7 | F8 | F9 | F10 |
|---|---|---|---|---|
| Lyo_T0 | >10 μm: 374 | >10 μm: 276 | >10 μm: 160 | >10 μm: 718 |
| | >25 μm: 30 | >25 μm: 10 | >25 μm: 26 | >25 μm: 92 |
| Lyo-25° C._2 h | >10 μm: 680 | >10 μm: 582 | >10 μm: 752 | >10 μm: 1182 |
| | >25 μm: 52 | >25 μm: 164 | >25 μm: 92 | >25 μm: 342 |
| Lyo-5° C._22 h | >10 μm: 2412 | >10 μm: 3794 | >10 μm: 1258 | >10 μm: 3050 |
| | >25 μm: 314 | >25 μm: 564 | >25 μm: 240 | >25 μm: 610 |

*Note:
Formulation was diluted 20 times with DI-water (1:20, v/v)
Conclusion

During preparation of Formulations, F9 had cloudy precipitation. After filtration, the assay test showed that the precipitation is the active (API). The results of particulate matter testing of F7, F8 and F10 showed that using water to make up the solutions was better than buffer. From the lyophilization results, it seemed that F8 with EDTA-Na2 and Methionine has no significant changes relative to F7. F7 was shown to be a better lead formulation if the formulation is lyophilized. F8 was shown to be the better lead formulation if the formulation is aqueous.

### Example 6: Administration of Pharmaceutical Formulations

A pharmaceutical formulation comprising a therapeutically effective amount of a lipid-based formulation of SEQ ID NO: 3 is administered to a subject in need thereof to treat or prevent a disease or condition. The administration can be by any appropriate route (e.g., injection, infusion, implantation).

A pharmaceutical formulation comprising a therapeutically effective amount of a lipid-based formulation of SEQ ID NO: 3 is administered to a subject in need thereof to regulate the complement system in the subject. The administration can be by any appropriate route (e.g., injection, infusion, implantation).

A pharmaceutical formulation comprising a therapeutically effective amount of SEQ ID NO: 2 or SEQ ID NO: 3 in combination with an excipient that is suitable for intravenous administration is intravenously administered to a subject in need thereof to treat or prevent a disease or condition. The excipient may be selected based on the age of the subject.

A pharmaceutical formulation comprising a therapeutically effective amount of SEQ ID NO: 2 or SEQ ID NO: 3 in combination with an excipient that is suitable for intravenous administration is intravenously administered to a subject in need thereof to regulate the complement system in the subject. The excipient may be selected based on the age of the subject.

### LIST OF EMBODIMENTS

The following is a non-exhaustive list of embodiments provided by the present description.
1. A composition comprising a therapeutically effective amount of SEQ ID NO: 3 and a lipid-based carrier.
2. The composition of embodiment 1, wherein the lipid-based carrier comprises lipid micelles.
3. The composition of embodiments 1 or 2, wherein the lipid-based carrier comprises a lipid emulsion.
4. The composition of embodiment 3, wherein the lipid-based carrier is present in an amount of about 10% w/v to about 20% w/v.
5. A composition comprising a therapeutically effective amount of SEQ ID NO: 2 or SEQ ID NO: 3 and at least one excipient.
6. The composition of embodiment 5, wherein the at least one excipient is suitable for intravenous administration.
7. The composition of embodiments 5 or 6, wherein the at least one excipient is selected from the group consisting of a citrate, an ascorbate, amino acids, and combinations thereof.
8. The composition of embodiment 7, wherein the citrate comprises sodium citrate.
9. The composition of embodiment 8, wherein the citrate is present in an amount of about 1% w/v to about 5% w/v.
10. The composition of embodiment 7, wherein the ascorbate comprises sodium ascorbate.
11. The composition of embodiment 10, wherein the ascorbate is present in an amount of about 1% w/v to about 5% w/v.
12. The composition of embodiment 7, wherein the amino acids comprise L-methionine.
13. The composition of embodiment 12, wherein the amino acids are present in an amount of about 0.01% w/v to about 5% w/v.
14. The compositions of any of embodiments 1-13, wherein SEQ ID NO: 2 or SEQ ID NO: 3 is present in an amount of about 0.001 to about 200 milligrams per kilogram (mg/kg) of body weight.
15. The composition of any of embodiments 1-14, wherein SEQ ID NO: 2 and/or SEQ ID NO: 3 is present in an amount of about 5 to about 160 mg/kg.
16. The composition of any of embodiments 1-13, wherein SEQ ID NO: 2 or SEQ ID NO: 3 is present in an amount of about 1 mg/ml to about 100 mg/ml.
17. The composition of any of claims 1-13, wherein SEQ ID NO: 2 or SEQ ID NO: 3 is present in an amount of about 10 mg/ml to about 80 mg/ml.
18. A method of altering cytokine expression comprising administering to a subject in need thereof a composition according to any of embodiments 1-17.
19. The method of claim 18, wherein the administration comprises parenteral administration.
20. The method of embodiment 18 or 19, wherein the administration comprises intravenous administration.
21. A method of treating or preventing a disease or condition comprising administering to a subject in need thereof a composition according to any of embodiments 1-17.
22. The method of embodiment 21, wherein the administration comprises parenteral administration.
23. The method of embodiment 21 or 22, wherein the administration comprises intravenous administration.

Sequence Table

| SEQ | | |
|---|---|---|
| ID | | |
| NO. | Sequence | Description |
| 1 | IALILEPICCQERAA | PA (PIC1) |
| 2 | IALILEPICCQERAA-PEG24 | PA-dPEG24 |
| 3 | IALILEP(Sar)CCQERAA | PA-I8Sar |

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

### REFERENCES

1. Fosgerau K and Hoffmann T (2014) Peptide therapeutics: current status and future directions. Drug Disc Today 20: 122-129.
2. Ali A M, Atmaj J, Van Oosterwijk N, Groves M R, Domling A (2019) Stapled peptide inhbitors: a new window for target drug discovery. Comp Struct Biotech J 17: 263-281
3. Sharp J A, Hair P S, Pallera H K, Kumar P S, Mauriello C T, et al. (2015) Peptide Inhibitor of Complement C1 (PIC1) Rapidly Inhibits Complement Activation after Intravascular Injection in Rats. PLoS ONE 10: e0132446.
4. Hair P S, Sass L A, Krishna N K, Cunnion K M (2017) Inhibition of Myeloperoxidase Activity in Cystic Fibrosis Sputum by Peptide Inhibitor of Complement C1 (PIC1). PLoS ONE 12: e0170203.
5. Hair P S, Cunnion K M, Krishna N K (2017) Peptide Inhibitor of Complement C1 Inhibits the Peroxidase Activity of Hemoglobin and Myoglobin. Int J Pept 2017: 9454583.
6. Gregory Rivera M, Hair P S, Cunnion K M, Krishna N K (2018) Peptide Inhibitor of Complement C1 (PIC1) demonstrates antioxidant activity via single electron transport (SET) and hydrogen atom transfer (HAT). PLoS ONE 13: e0193931.
7. Hair P S, Enos A I, Krishna N K, Cunnion K M (2018) Inhibition of Immune Complex Complement Activation and Neutrophil Extracellular Trap Formation by Peptide Inhibitor of Complement C1 Front Immunol 9: 558.
8. Hair P S, Rivera M G, Enos A I, Pearsall S E, Sharp J A, et al. (2017) Peptide Inhibitor of Complement C1 (PIC1) Inhibits Growth of Pathogenic Bacteria. International Journal of Peptide Research and Therapeutics DOI 101007/s10989-017-9651-z.
9. Matsui S M, Kiang D, Ginzton N, Chew T, Geigenmuller-Gnirke U (2001) Molecular biology of astroviruses: selected highlights. Novartis Found Symp 238: 219-233; discussion 233-216.
10. Bonaparte R S, Hair P S, Banthia D, Marshall D M, Cunnion K M, et al. (2008) Human astrovirus coat protein inhibits serum complement activation via C1, the first component of the classical pathway. J Virol 82: 817-827.
11. Hair P S, Enos A I, Krishna N K, Cunnion K M. (2019) Inhibition of complement activation, myeloperoxidase, NET formation and oxidant activity by PIC1 peptide variants. PLoS ONE 14: e0226875.
12. Cunnion K M, Lee J C, Frank M M (2001) Capsule production and growth phase influence binding of complement to Staphylococcus aureus. Infect Immun 69: 6796-6803.

1. A composition comprising a therapeutically effective amount of SEQ ID NO: 3 and a lipid-based carrier.

2. The composition of claim 1, wherein the lipid-based carrier comprises lipid micelles.

3. The composition of claim 1, wherein the lipid-based carrier comprises a lipid emulsion.

4. The composition of claim 3, wherein the lipid-based carrier is present in an amount of about 10% w/v to about 20% w/v.

5. A composition comprising a therapeutically effective amount of SEQ ID NO: 2 or SEQ ID NO: 3 and at least one excipient.

6. The composition of claim 5, wherein the at least one excipient is suitable for intravenous administration.

7. The composition of claim 5, wherein the at least one excipient is selected from the group consisting of a citrate, an ascorbate, amino acids, and combinations thereof.

8. The composition of claim 7, wherein the citrate comprises sodium citrate.

9. The composition of claim 8, wherein the citrate is present in an amount of about 1% w/v to about 5% w/v.

10. The composition of claim 7, wherein the ascorbate comprises sodium ascorbate.

11. The composition of claim 10, wherein the ascorbate is present in an amount of about 1% w/v to about 5% w/v.

12. The composition of claim 7, wherein the amino acids comprise L-methionine.

13. The composition of claim 12, wherein the amino acids are present in an amount of about 0.01% w/v to about 5% w/v.

14. The compositions of claim 1, wherein SEQ ID NO: 2 or SEQ ID NO: 3 is present in an amount of about 0.001 to about 200 milligrams per kilogram (mg/kg) of body weight.

15. The composition of claim 1, wherein SEQ ID NO: 2 and/or SEQ ID NO: 3 is present in an amount of about 5 to about 160 mg/kg.

16. The composition of claim 1, wherein SEQ ID NO: 2 or SEQ ID NO: 3 is present in an amount of about 1 mg/ml to about 100 mg/ml.

17. The composition of claim 1, wherein SEQ ID NO: 2 or SEQ ID NO: 3 is present in an amount of about 10 mg/ml to about 80 mg/ml.

18. A method of altering cytokine expression comprising administering to a subject in need thereof a composition according to claim 1.

19. The method of claim 18, wherein the administration comprises parenteral administration.

20. The method of claim 18, wherein the administration comprises intravenous administration.

21. A method of treating or preventing a disease or condition comprising administering to a subject in need thereof a composition according to claim 1.

22. The method of claim 21, wherein the administration comprises parenteral administration.

23. The method of claim 21, wherein the administration comprises intravenous administration.


SEQUENCE LISTING (3 sequences)

SEQ ID NO: 1 - protein, 15 aa, Artificial Sequence
  Description of Artificial Sequence Synthetic peptide
IALILEPICCQERAA

SEQ ID NO: 2 - protein, 15 aa, Artificial Sequence
  Description of Artificial Sequence Synthetic peptide
  C-term PEG24
IALILEPICCQERAA

SEQ ID NO: 3 - protein, 15 aa, Artificial Sequence
  Description of Artificial Sequence Synthetic peptide
  MOD_RES (8)..(8): Sarcosine
IALILEPXCCQERAA